(19) United States
(12) Patent Application Publication
(10) Pub. No.: US 2011/0004259 A1
(43) Pub. Date: Jan. 6, 2011

(54) REFERENCE FRAME FIXATOR

(75) Inventors: Jody Stallings, Palm Harbor, FL (US); Alfred Tria, Princeton, NJ (US)

Correspondence Address: DIANA HOUSTON;SMITH & NEPHEW, INC., 1450 BROOKS ROAD, MEMPHIS, TN, 38116, US

(73) Assignee: SMITH & NEPHEW, INC., Memphis, TN (US)

(21) Appl. No.: 12/515,398

(22) PCT Filed: Nov. 26, 2008
(86) PCT No.: PCT/US08/84992

§ 371 Date: Sep. 23, 2010

Related U.S. Application Data
(60) Provisional application No. 60/866,316, filed on Nov. 17, 2006.

(51) Int. Cl. A61B 17/56

(52) U.S. Cl. 606/86 R

(57) ABSTRACT

A device for positioning a fiducial marker (160) on an anatomical structure includes a fiducial base (40) and a fixation member (60). The fiducial base (40) comprises a turn (44) and an extension (42) configured to position the fiducial marker (160) within the field of view of a tracking sensor (210). The fiducial marker (160) is positioned away from the anatomical structure. The fixation member (60) is configured to have a low profile and further configured to fix the fiducial base (40) to the anatomical structure. The fixation member (60) is fixed to the anatomical structure through a primary surgical incision and positioned on the anatomical structure such that the fixation member (60) is isolated from the surgical approach. The fiducial base (40) extends from the fixation member through the primary surgical incision.

### CROSS-REFERENCE TO RELATED APPLICATIONS

This application claims priority to U.S. Provisional Patent Application No. 60/866,316 filed on Nov. 17, 2006. The disclosure of this prior application is incorporated by reference in its entirety.

### BACKGROUND

1. Field

The present invention relates generally to computer assisted surgery and more particularly to reference frames for capturing positions in computer assisted surgery.

2. Related Art

Many reference frames are fixed to a patient through percutaneous pins placed through the quad muscles into the tibia. The reference frames are attached to tracking devices for surgical navigation. Because the reference frames are directly fixed to the bone through the percutaneous pins, the size and depth of the pins may cause stress risers in the bone. In addition, when using percutaneous pins, there is a potential to hit nerves, arteries and other structures resulting in injury, as well as introduce additional openings for infection. Moreover, the use of percutaneous pins may also block the intramedullary (IM) canal, which may cause problems in fixation if a prosthesis uses an IM fixator, or may cause problems if additional alignment through the IM canal is used for component placement and bone resection guidance.

The reference frame is generally fixed to bone away from the surgical site. For example, in a replacement knee surgery, the femur is located within the computer system using a reference frame superior to the knee joint. The tibia is referenced through a reference frame inferior to the knee joint. By locating the reference frames superior and inferior to the joint, the reference frames may be isolated from the surgical zone so that exposure within the joint is maximized without additional tools being placed within the initial skin cut. However, as previously noted, the placement of the reference frame superior or inferior to the joint creates additional problems through stress risers, soft tissue injuries, additional sites of possible infection, and IM canal blockage.

### SUMMARY

A device may provide for positioning a fiducial marker on an anatomical structure. The device includes a fiducial base and a fixation member. The fiducial base comprises a turn and an extension configured to position the fiducial marker within the field of view of a tracking sensor. The fiducial marker is positioned away from the anatomical structure. The fixation member is configured to have a low profile and further configured to fix the fiducial base to the anatomical structure. The fixation member is fixed to the anatomical structure through a primary surgical incision and positioned on the anatomical structure such that the fixation member is isolated from the surgical approach. The fiducial base extends from the fixation member through the primary surgical incision.

A method may be provided for fixing a fiducial marker to an anatomical structure. The fiducial marker is registered in a computer assisted surgical system. The method accesses the anatomical structure through a primary surgical incision. A fixation member is positioned on the anatomical structure. The anatomical structure is positioned such that the structure does not disturb operating surfaces within the surgical incision. The method slides a fiducial base into the fixation member. The fiducial base extends the fiducial marker away from the surgical incision such that the fiducial marker is positioned in the field of view of a tracking sensor of the computer assisted surgical system.

A low profile bone fixation member for a fiducial marker may be provided. The bone fixation member includes a positioning member, a guide and a bias member. The positioning member is configured to secure the bone fixation member to an anatomical structure. The guide is configured to slidably receive the fiducial marker. The guide is configured to slidably receive the fiducial marker in a direction generally perpendicular to the positioning member. The bias member is configured to secure the fiducial marker slidably received by the guide to the guide.

A computer assisted surgical system may include a bone fixation member, a fiducial marker, a tracking sensor, a guide, and a processor. The low profile bone fixation member comprises a positioning member configured to secure the bone fixation member to an anatomical structure. The fiducial marker is configured to attach to the bone fixation member and fix positional information about the anatomical structure. The tracking sensor is configured to receive positional information from the fiducial marker. The guide is configured to slidably receive the fiducial marker. The guide is configured to slidably receive the fiducial marker in a direction generally perpendicular to the positioning member. The processor configured to calculate real positions of bones from the positional information.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 is a view of an embodiment of a fiducial base according to an aspect of the invention;

FIG. 2 is a view of another embodiment of a fiducial base according to an aspect of the invention;

FIG. 3 is a view of an embodiment of the fiducial base of FIG. 2 and an embodiment of a bone fixation member according to an aspect of the invention;

FIG. 4 is a view of a pair of fixation members coupled to a femur and a tibia;

FIG. 5 is a view of the fiducial bases of FIGS. 1 and 2 coupled to the bone fixation members of FIG. 4;

FIG. 6 is a cross sectional view of an embodiment of a fiducial base coupled to a bone according to an aspect of the invention;

FIG. 7 is a view of an embodiment of a fiducial base and bone fixation member coupling a fiducial marker to a bone according to an aspect of the invention; and

FIG. 8 is a schematic view of a computer assisted surgical system according to an aspect of the invention.

### DETAILED DESCRIPTION OF THE EMBODIMENTS

The following description of the preferred embodiment(s) is merely exemplary in nature and is in no way intended to limit the invention, its application, or uses.

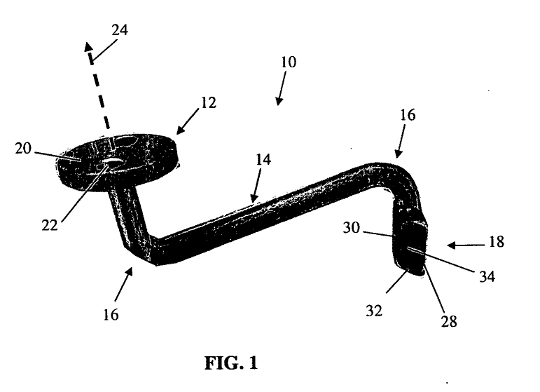

Turning now to FIG. 1, FIG. 1 is a view of an embodiment of a fiducial base 10 according to an aspect of the invention. The fiducial base 10 includes a marker platform 12, an extending arm 14, turns 16, and a male base fixation member 18. The marker platform 12 is configured to couple a fiducial marker to the fiducial base 10. The extending arm 14 spaces the fiducial marker attached to the marker platform 12 away from the male base fixation member 18 according to the number, direction, and degrees of the turns 16. The male base fixation member 18 couples the fiducial base 10 to a bone fixation member, which then couples the fiducial base 10 to a bone.

The extending arm 14 and the turns 16 may be sized according to a desired end position of the fiducial marker. By specifying the length of the extending arm 14 and the placement, direction, and degrees of the turns 16, the fiducial base 10 may position the fiducial marker in a desired position relative to the working area of the surgery and still within the field of vision of the computer assisted surgical system. In an alternative embodiment, the extending arm 14 and turns 16 may include a continuous turn such that no part of the extension is primarily straight. While the extending arm 14 and the turns 16 generally extend the reference frame and avoid extending into the working area of the surgeon, any shape of the extending arm 14 and turns 16 may be used. The computer assisted surgical system may account for different shapes of the fiducial base 10 when the shape of the fiducial base 10 is stored within the computer assisted surgical system.

The marker platform 12 is configured to attach to a fiducial marker. In one embodiment, as shown in FIG. 1, the marker platform 12 has a circular mating surface 20. Recesses 22 within the circular mating surface 20 receive the fiducial marker. The marker platform 12 may have magnets, such as a neodymium magnet, within the recesses 22 so that the fiducial marker may be coupled to the fiducial base 10 with positive fixation from the force bias created by the magnets. In addition, the fiducial marker may also include magnets on posts configured to couple with the recesses 22. The posts on the fiducial marker would be oppositely polarized from the magnets within the recesses 22. Moreover, in order to specify a certain orientation in the fiducial marker, magnets within the recesses 22 may be oppositely polarized from one another such that the fiducial marker would only achieve positive fixation in a single orientation of the fiducial marker with respect to the fiducial base 10.

In addition to using magnets to orient and fix the fiducial marker relative to the fiducial base 10, the marker platform 12 may have differently shaped recesses 22 so that the fiducial marker may fit in a specific orientation. In another embodiment, the mating surface 20 may have a roughened surface so that the fiducial marker is less likely to slip relative to the fiducial base 10.

In order for the fiducial marker to be properly viewed within the field of view of the surgical system and properly calculate the position of the bone to which the fiducial base 10 is attached, the fiducial marker may need to be rotated relative to an axis 24 perpendicular to the marker platform 12. Any rotation of the fiducial marker around this axis 24, as long as the rotation is completed before registration of the fiducial marker and not changed after registration, may fix the relative position of the fiducial marker to the bone for accurate computer visualization of the anatomy. The mechanism to rotate the fiducial marker relative to the marker platform 12 may be positioned either at the marker platform 12 or on the fiducial marker.

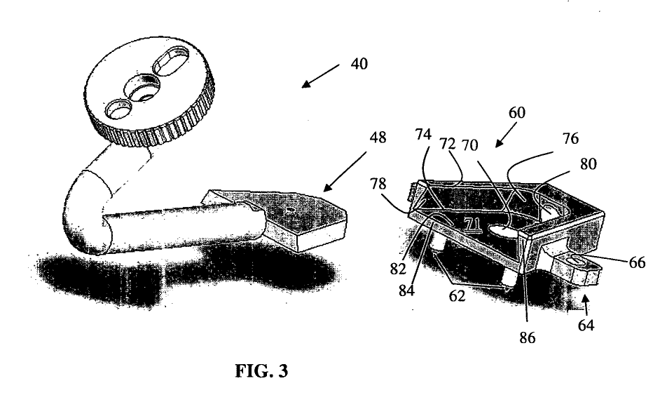

The male base fixation member 18 is configured to fix the fiducial base 10 to a female bone fixation member (as shown in FIG. 3, and discussed below) which attaches to a bone. The male base fixation member 18 includes a lower mating edge 28, an upper mating edge 30, a forward mating surface 32, a side mating surface 34, and a rear surface portion 36. The male base fixation member 18 is configured to slide into a recess in the female bone fixation member. The forward mating surface 32 is configured to mate with a forward surface of the female bone fixation member.

In this embodiment, the side mating surface 34 is a beveled surface from the lower edge 28 to the upper edge 30, and also a beveled surface from the rear surface portion 36 to the forward mating surface 32. The double beveled side mating surface 34 allows for initial gross positioning of the fiducial base 10 which transitions to fine positioning as the male base fixation member 18 is seated fully in the female bone fixation member. Alternatively, other embodiments may include a single beveled side mating surface, or a beveled side mating surface which has a double beveled surface from the lower edge 28 to the upper edge 30. The bevels in the mating surface allows for an operator to generally align the fiducial base 10 into the female bone fixation member. As the fiducial base 10 is advanced into the female bone fixation member, the beveled surfaces guide the fiducial base 10 into alignment in the base.

In one embodiment, when the male base fixation member 18 is seated, a magnet positively biases the male base fixation member 18 into the female bone fixation member. The magnet may be positioned within the male base fixation member 18 or the female bone fixation member, or both. The magnet(s) creates a magnetic force between the base fixation members to hold the base fixation members together. The force allows for small perturbations of the fiducial base 10 without dislodging the fiducial base 10 from the female bone fixation member. In addition, the small bias force also allows for a large perturbation (such as strongly knocking the fiducial marker, or purposely pulling on the marker) to dislodge the fiducial base 10 from the female bone fixation member without pulling the female bone fixation member from the bone. Such a system, then, creates a mechanical weak point at the fixation members to protect the bone from damage.

In addition to magnets, other positive bias forces may be used to retain the male base fixation member 18 (and thus the fiducial base 10) within the female fixation base member. Mechanical locking systems, which may be mechanically released, may be used to positively bias the male base fixation member 18 to the female bone fixation member. If the mechanical locking system is not releasable, then the locking mechanism may be made weaker than the other systems affixing the fiducial to the bone so that when the surgical procedure is completed, the fiducial base 10 may be removed by breaking the mechanical locking system. Such breakable locking systems may be single-use, disposable systems.

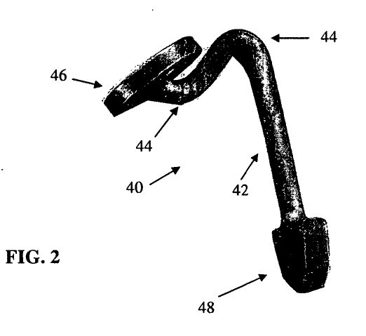

Turning now to FIG. 2, FIG. 2 is a view of another embodiment of a fiducial base 40 according to an aspect of the invention. The base 40 includes an extending arm 42, turns 44, a marker platform 46, and a male base fixation member 48. The components 42-48 of the fiducial base 40 are similar to components of the fiducial base 10 of FIG. 1. The platform 46 is configured to support and fix a fiducial marker to the base 40. The male base fixation member 48 is configured to orient and fix the base 40 to the female bone fixation member and thus fix the base 40 to the bone. Similar to the embodiment of FIG. 1, the extending arm 42 and the turns 44 are configured to space the platform 46 from the male base fixation member 48.

The turns 44 and the extending arm 42 of the base 40 of FIG. 2 are shaped differently than the turns and extending arms of the base of FIG. 1. The different lengths of the extending arms 42, placement of turns 44, and degrees of the turns 44 orient the platform 46 relative to the male base fixation member 48 in placement different from the platform and the male base fixation member of FIG. 1. Such different orientations between the platform 46 relative to the male base fixation member 48 allow the base 40 to extend the fiducial markers in positions that minimally encroach the surgical area while maintaining the fiducial markers within the field of vision of the computer assisted surgical system.

Turning now to FIG. 3, FIG. 3 is a view of an embodiment of the fiducial base 40 of FIG. 2 and an embodiment of a female bone fixation member 60 according to an aspect of the invention. The female bone fixation member 60 includes spikes 62, attachment arms 64, and screw recesses 66 and 70. A guide 71 of the female bone fixation member 60 includes an upper edge 72 a lower edge 74, a forward mating surface 76 and a side mating surface 78. A bias member 80 may be located on the forward mating surface 76. A lower surface 82 is defined by a rear edge 84 and the lower edge 74. The side mating surface 78 is defined vertically by the upper edge 72 to the lower edge 74 and horizontally from each side edge 86 to the front mating surface 78. The side mating surface 78 is configured to mate with the beveled surface of the male bone fixation member 48.

The bevels in the female bone fixation member 60 are oriented to receive the male fixation member 48. The female bone fixation member 60 is beveled from the rear edge 84 to the forward mating surface 76, and is further beveled from the lower edge 74 to the upper edge 72. In addition to these compound bevels, it may be desirable to additionally bevel the male and female fixation members 40 and 60 with a bevel where the upper edge 72 and lower edge 74 converge as the upper edge 72 and lower edge 74 are traced from the rear edge 84 toward the forward mating surface 76. The bevels may allow for initial gross placement of the male fixator 40 so that an operator may initially align the male fixator 40 with the female fixator 60. Such a configuration allows for an operator to be able to confidently place the fixators 40 and 60 in obstructed or reduced views by “feeling” for contact between the male and female fixators 40 and 60. A generally tapered shape to the male fixator 40 allows for this general gross placement of the fiducial base.

In this embodiment, the bias member 80 may use magnets, such as a neodymium magnet, within a recess in the forward mating surface 76 so that the male fixation base member 40 may be coupled to the female bone fixation member 60 with positive fixation from the force bias created by the magnet. In alternate embodiments, the magnet may be positioned within the male base fixation member 40 or the female bone fixation member 60, or both. The magnet(s) creates a magnetic force between the base fixation members to hold the base fixation members 40 and 60 together. The force allows for small perturbations of the fiducial base without dislodging the fiducial base from the female bone fixation member 60. In addition, the small bias force also allows for a large perturbation to dislodge the fiducial base from the female bone fixation member 60 without pulling the female bone fixation member 60 from the bone by ripping out the screws that attach the female bone fixation member 60 to the bone through the screw holes 66 and 70. Such a system, then, creates a mechanical weak point at the fixation members 40 and 60 to protect the bone from damage from screw pullout.

The screw recesses 66 and 70 receive small screws to attach the female bone fixation member 60 to the bone. The recesses 66 and 70 may be used alternatively, or together, depending upon the placement of the female bone fixation member 60. The external screw recesses 66 may provide a lower profile for the female bone fixation member 60 because the head of the screw used to affix the female bone fixation member 60 to the bone does not need to be fully seated within the guide 71 of the female bone fixation member 60. By utilizing the recess 70 to screw the female bone fixation member 60 to the bone, the screw head of the affixing screw must be generally flush with the lower surface 82 of the female bone fixation member 60 in order to receive the male base fixation member 40.

While the recesses 66 and 70 generally fix the female bone fixation member 60 to the bone, the spikes 62 set the female bone fixation member 60 to the bone. The spikes 62, generally perpendicular to the lower surface 82 of the guide 71, are first set in the bone to orient the female bone fixation member 60 relative to the bone. The spikes 62 are pressed or punched into the bone, for example, by a hammer. The spikes 62 initially orient the female bone fixation member 60 so that the operator may check to verify the angles of the female bone fixation member 60 and the male base fixation member 48 are properly aligned within the field of view of the tracking system prior to permanent fixation of the female bone fixation member 60 to the bone. Once the operator is satisfied with the initial setup with the spikes 62, then the operator may screw the access screws through the recesses 66 and 70 to set the female bone fixation member 60 to the bone.

The spikes 62 and screws are generally short. The small size of the spikes 62 and screws allows for placement of fiducials which do not block the IM canal. Longer spikes or screws would extend through the bone into the IM canal, which would block the IM canal and interfere with placement of additional alignment devices through the IM canal, such as the alignment devices commonly used in total knee replacement surgeries. The smaller spikes 62 and the screws may not need to be as deep into the bone as the screws because the geometry and characteristics of the base 40 allow for lower transmitted forces and moments to the fixation member 60.

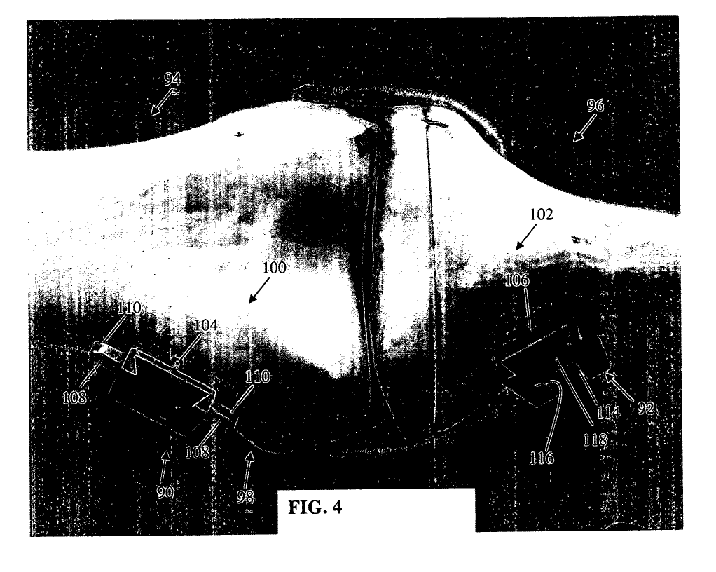

Turning now to FIG. 4, FIG. 4 is a view of a pair of fixation members 90 and 92 coupled to a femur 94 and a tibia 96. Fixation member 90, may be attached to the femur 94 proximal to the medial epicondyle 98 and posterior to the adductor tubercle 100. Such a placement would allow the femoral fixation member 90 to be fixed to the bone through the primary incision for the surgical procedure, for example a total knee replacement. The fixation member 92 may be placed distal, medial and posterior to the tibial tuberosity 102. Similarly, this placement also allows the tibial fixation member 92 to be inserted through the primary incision.

Both the femoral fixation member 90 and the tibial fixation member 92 are placed to minimize operator interference, particularly from interfering with a surgeon. By placing the fixation members 90 and 92 medial to the center of the joint, the surgeon or other technicians may not cross over the fiducials in order to access the joint. Moreover, the placement of the femoral fixation member 90 proximal to the joint working surfaces and the placement of the tibial fixation member 92 inferior to the joint working surfaces also minimizes interference between the fiducials and a surgeon.

The base fixation members 90 and 92 are oriented to project the fiducial bases toward the joint. Such an orientation minimizes the need to increase the incision size by allowing for the fiducial bases to project toward the joint while at the same time projecting anterior to the joint to move the bases away from the working area of the joint. The base fixation members 90 and 92 may be oriented to project the fiducial bases close to the ends of the incision, or may be oriented more toward the middle of the incision.

The base fixation members 90 and 92 are initially set in the bone with spikes 104 and 106. The spikes 104 and 106 are spaced differently than the spikes from the fixation member of FIG. 3. The spikes 104 and 106 are spaced from the front to the back of the base fixation members 90 and 92, while the spikes of FIG. 3 are placed laterally from side to side. Other embodiments, including spacing the spikes both from side to side and from front to back may be utilized according to the position of fixation and user preference.

The femoral fixation member 90 is fixed to the bone using screw recesses 108 through attachment arms 110. The attachment arms 110 may be located on the sides or on either the front end or the rear end of the fixation member 90. The arms 110 may be offset from one another along the length of the sides or the ends, or may be positioned one on a side and one on an end. While this embodiment has shown a pair of attachment arms 110, it may be beneficial to use a single attachment arm or more than two attachment arms according to the anatomy of the placement or the preference of the surgeon. Using a single attachment arm, the spikes 104 may help fix the fixation member 90 to the bone without rotation of the fixation member 90 about the screw recess 108.

The tibial fixation member 92 is configured with an open front end 114. The forward movement of the base into the fixation member 92 is controlled by beveled side surfaces 116 of the fixation member 92. While both sides of the fixation device 92 are beveled, it may be possible to have only a single side of the fixation member 92 beveled. In addition, the tibial fixation member 92 further includes a single center screw hole 118 to affix the fixation member 112 to the bone. The additional thickness of the fixation member 92 compared to the thickness of the fixation member 90 may be attributed to the center screw hole 118 which may flush the screw relative to the fixation member 92.

While the different fixation members 90 and 92 of FIG. 4 and the fixation member 60 of FIG. 3 have different features and alternative means for achieving different functions, the different features and alternates may be mixed and modified across different fixation members both as shown in the drawings and as discussed above. For example, an open-ended fixation member like fixation member 92 may have a pair of attachment arms like the fixation member 90, where the attachment aims are offset along the side walls of the fixation member.

Other fixation members may also be configured with male mating portions. In such an embodiment, the fiducial base may be configured having a female mating portion. Such a configuration may allow for the bone fixation member to have a low profile for improved ease of implantation when the bone fixation member is affixed to the bone. In addition, a male bone fixation member may have beveled edges as previously described. Generally, the mating portions of the bone fixation member and the fiducial base are negatives of each other, such that the negative spaces of the female mating portion is shaped like the male mating portion, and vice versa.

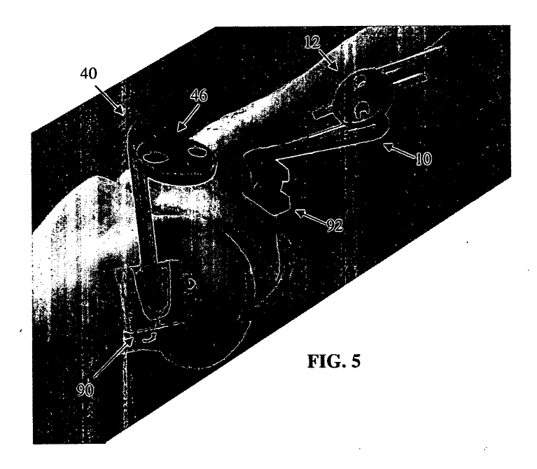

Turning now to FIG. 5, FIG. 5 is a view of the fiducial bases 10 and 40 of FIGS. 1 and 2 coupled to the bone fixation members 90 and 92 of FIG. 4. The fiducial base 10, attached to the tibial bone fixation member 92, extends toward the inferior portion of the knee joint, where the inferior portion of an incision would be located. The fiducial base 40 attached to the femoral bone fixation member 90 extends toward the knee joint from the superior location of the femoral bone fixation member 90 where the superior portion of a skin incision would be located.

The orientation of the bases 10 and 40 and bone fixation members 90 and 92 ease access to the joint while keeping the fiducials in the field of view. The bases 10 and 40 extend anterior to the knee joint, which may allow the bases 10 and 40 and the markers connected to the bases 10 and 40 to be elevated away from the working area around the knee joint. In addition, in this embodiment, the planes of the surfaces of the platforms 12 and 46 of the bases 10 and 40 are not parallel when the knee joint is fully extended. However, as the knee is flexed, the planes of the surfaces of the platforms 12 and 46 of the bases 10 and 40 rotate into more parallel orientations. This may allow fiducials, extending perpendicular to the planes of the surfaces of the platforms 12 and 46 to also be parallel and jointly viewable within the field of view of the computer assisted surgical system. Moreover, the projections of the bases 10 and 40 may minimize loss of data caused by obstruction between the sensing system and the fiducials from either an operator or the patient by elevating the fiducials away from the working area of the joint. Other embodiments which plan for the relative placement of the fiducials with respect to the sensing system of the computer assisted surgical system may use differently oriented bone fixation members 90 and 92, or differently shaped bases 10 or 40 according to the field of view of the computer assisted surgical system.

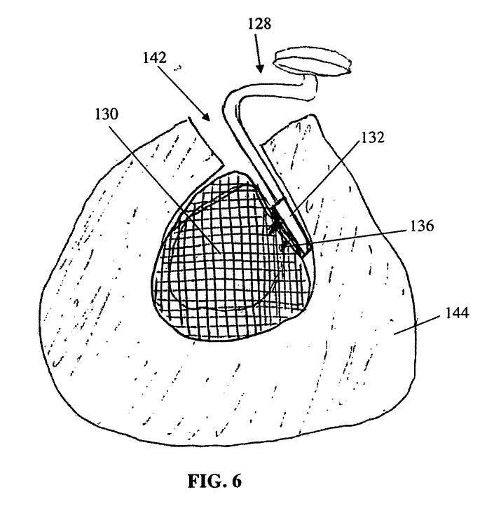

Turning now to FIG. 6, FIG. 6 is a cross sectional view of an embodiment of a fiducial base 128 coupled to a bone 130 according to an aspect of the invention. A bone fixation member 132 is set into the bone 130 using spikes 136. The fiducial base 128 is inserted into the bone fixation member 132 and extends toward an incision 142 in soft tissue 144, including skin and muscle. The bone fixation member 132, then, is located between the soft tissue 144 and the bone 130. The soft tissue 144 may also add a slight pressure to the bone fixation member 132 to fix the bone fixation member 132 to the bone 130.

When the bone fixation member 132 and the base 128 are installed, the surgeon begins by first making the incision necessary for the surgery. Thus, the installation of the bone fixation member 132 may not require a longer incision. The soft tissue 144 is pulled back to expose as much of the bone 130 as possible. The surgeon may slide the bone fixation member 132 under the soft tissue 144. The bone fixation member 132 may be attached to the bone 130 with a small mallet or other device used to impart a direct force to the bone fixation member 132, driving the spikes 136 into the bone. The surgeon may then check the orientation of the bone fixation member 132 by inserting the base 128 into the bone fixation member 132. If the orientation is correct, then the surgeon may use the small screws to attach the bone fixation device 132 to the bone 130. If the orientation is not correct, then the surgeon may reset the bone fixation member 132, or may try additional bases that are shaped differently. Once the bone fixation device 132 is properly oriented and fixed to the bone 130, then the fiducial base 128 is set in the bone fixation member 132.

When the bone fixation member 132 is fixed under the soft tissue 144 to the bone 130, the bone fixation member 132 may be accessed “blind.” It may not be necessary for the surgeon to see the bone fixation member 132 when inserting the base 128 into the bone fixation member 132. The beveled surfaces of the bone fixation member 132 and the base 128 allow for a surgeon to first insert the smaller male mating portion of the base 128 into the largest female mating portion bone fixation member 132. Thus, the base 128 is guided into the bone fixation member 132 by feel.

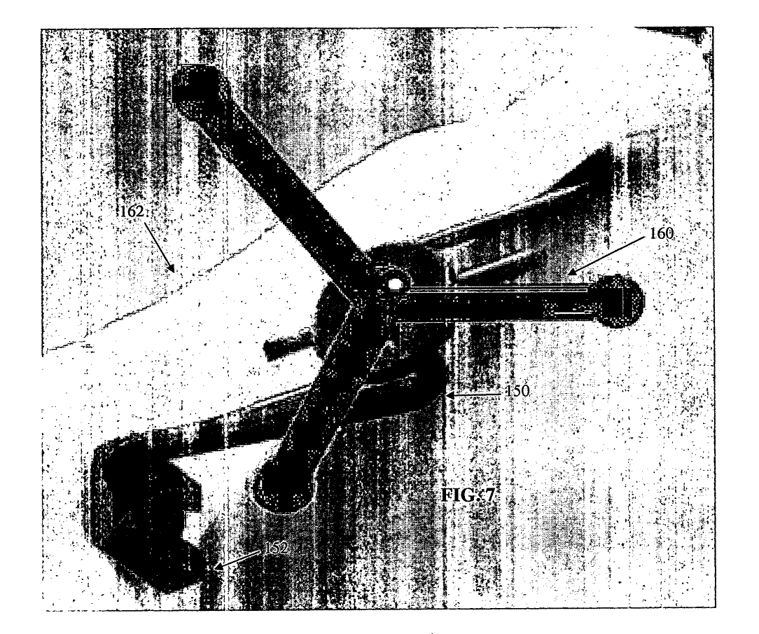

Turning now to FIG. 7, FIG. 7 is a view of an embodiment of a fiducial base 150 and bone fixation member 152 coupling a fiducial marker 160 to a bone 162 according to an aspect of the invention. The fiducial marker 160 is extended above the joint and medial to the joint so that a surgeon operating from the lateral side of the joint is less likely to come into contact with the fiducial marker 160 or any of the other parts of the system.

The bone fixation member 152, as previously stated above, may be designed for fixating to the bone in an obstructed view. The bevels in the mating surfaces of the bone fixation member 152 allows for gross placement of the fiducial base 150 into the bone fixation member 152, which when further slid along the mating surfaces, fixes the fiducial base 150 into the bone fixation member 152. In addition, the bone fixation member 152 allows for fixation of the fiducial marker 160 without invading the IM canal without using long screws to fix the fiducial marker 160 to the bone 162.

The fiducial base 150 is configured to position the fiducial marker 160 away from the bone 162. The fiducial base 150 may use a combination of turns and extensions to extend the fiducial marker 160 away from the bone 162 through the primary incision, and elevated from the working area of the surgery. Depending on the number of turns, degree of the turns, and length and placement of the extensions, varying geometries may be achieved to position the fiducial marker 16 away from the surgical approach.

The bone fixation member 152 is positioned to minimally interfere with the surgical approach. This requires placement of the bone fixation member 152 away from the surgical incisions. The bone fixation member 152, then, is isolated from interfering with tools in surgery. For example, a more centrally placed fiducial may use long screws to attach the fiducial to the bone. In such a system, bone cuts and guide placement may be affected by the long screws, or even the fiducial itself. When the bone fixation member 152 is attached to the bone 162, its low profile and gross guides may allow for positioning farther from the surgical approaches. Moreover, the structure of the fiducial base 150 extending through the incision farther from the more centralized portions of the surgical approach may minimize obstructions caused by the fiducial marker during surgery.

In addition, the connections between the fiducial base 150 and either the bone fixation member 152 or the fiducial marker 160 may be detachable. A detachable connection may allow for small perturbations of the fiducial marker 160 or the fiducial base 150 without dislodging the bone fixation member 152 from the bone 162 or adjusting the bone fixation member 152 relative to the bone 162. When the marker 160 is disturbed, the connections may detach, which would then require an operator to reconnect the detached connection. For example, a slight perturbation may dislodge the marker 160 from the fiducial base 150. Minimal forces would be transferred to the bone fixation member 150, and thus preserve the bone fixation member 152 to bone 162 connection.

The devices 150-160 may be fabricated from rigid biocompatible material. The material may be worked using standard CNC machining processes, or by other manufacturing processes. Magnets, placed in the connections between the devices 150-160, may be made from rare earth metal materials such as neodymium. Sensors in the fiducial markers 160 may be made from a material detectable in the field of view of the computer assisted surgical system, and preferentially, the material for the sensors should be different from the material which makes the fiducial base 150 the bone fixation member 152 and the frame portion of the fiducial marker 160 so that the sensors are primarily viewable by a sensor within the computer assisted surgical system.

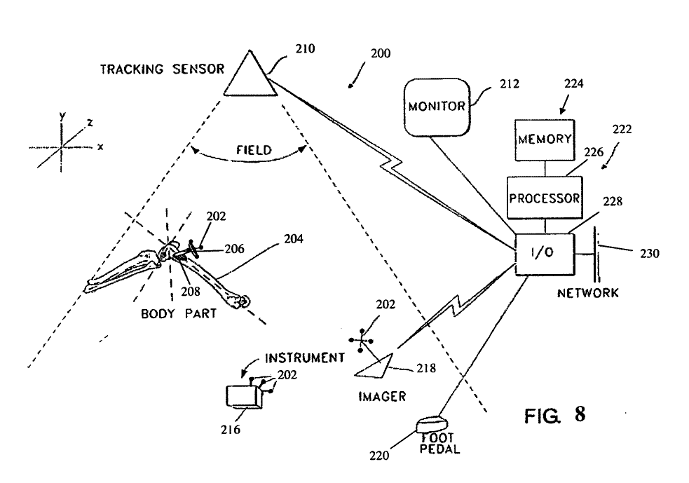

Turning now to FIG. 8, FIG. 8 is a schematic view of a computer assisted surgical system 200 according to an aspect of the invention. The computer assisted surgical system 200 uses a fiducial marker 202 to obtain the position and orientation of a bone 204 when the marker 202 is distanced from the bone 204 using a fiducial base 206 and a bone fixation member 208. A tracking sensor 210 images the marker 202 such that a representation of the bone 204 may be displayed on a monitor 212. Other fiducial markers 202 may be placed on instruments 216 so that these devices may also be displayed on the monitor 212. An imager 218 may be used to correlate the information from the markers 202. A foot pedal 220, controlled by an operator such as the surgeon may also be used in the computer assisted surgical system 200 for input. Input from the tracking sensor 210, the imager 218 and the foot pedal 220 may be input into a computer interface 222 for collection and processing to output to the monitor 212. The computer interface 222 may include memory 224, a processor 226 and an input/output interface 228. The i/o interface 228 may also be connected to a network 230, for transmission over a network to other individuals or other storage mediums.

When the system is initiated, the marker 202 is registered at a first position of the bone 204. As the bone 204 is moved, the marker 202 is moved and rotated in three dimensions relative to the first position. By using additional markers attached to the bone 204, the computer interface 222 may calculate positions of the bone 204, because the marker 202 does not move or rotate relative to the bone 204. Additionally, the computer interface 222 may also register instruments 216 within the field of the tracking sensor 210 so that instrument movement may also be tracked.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A device for positioning a fiducial marker on an anatomical structure, comprising: a. a fiducial base comprising a turn and an extension configured to position the fiducial marker within the field of view of a tracking sensor, the fiducial marker being positioned away from the anatomical structure; and b. a fixation member configured to have a low profile and further configured to fix the fiducial base to the anatomical structure, the fixation member being fixed to the anatomical structure through a primary surgical incision and being positioned on the anatomical structure such that the fixation member is isolated from the surgical approach; the fiducial base extending from the fixation member through the primary surgical incision.

2. The device of claim 1, wherein the fixation member comprises a temporary positioning member comprised to temporarily attach the fixation member to the anatomical structure.

3. (canceled)

4. (canceled)

5. The device of claim 2 further comprising a permanent position member configured to fix the fixation member to the anatomical structure.

6. The device of claim 5, wherein the permanent position member comprises an arm comprising a screw hole configured to receive a screw for screwing the fixation member to the anatomical structure.

7. The device of claim 5, wherein the fixation member further comprises a mating portion, and the fiducial base further comprises a complementary mating portion, the mating portion and the complementary mating portion being negatives of each other.

8. (canceled)

9. (canceled)

10. The device of claim 7 wherein the mating portion further comprises a beveled surface and the complementary mating portion further comprises a complementary beveled surface, the beveled surface configured to mate with the complementary beveled surface.

11. (canceled)

12. (canceled)

13. (canceled)

14. (canceled)

15. The device of claim 1, wherein the fixation member further comprises a mating portion, and the fiducial base further comprises a complementary mating portion, the mating portion and the complementary mating portion being negatives of each other.

16. (canceled)

17. The device of claim 15, wherein the mating portion is configured to receive the complementary mating portion in a first direction, the first direction being configured to align along an axis of the mating portion.

18. The device of claim 15, wherein the mating portion further comprises a beveled surface and the complementary mating portion further comprises a complementary beveled surface, the beveled surface configured to mate with the complementary beveled surface.

19. The device of claim 18, wherein the mating portion further comprises a second beveled surface and the complementary mating portion further comprises a second complementary beveled surface wherein the beveled surface and the second beveled surface form a compound beveled surface, the second beveled surface configured to mate with the second complementary beveled surface.

20. The device of claim 15, wherein the fiducial base further comprises a platform configured to couple the fiducial marker to the fiducial base.

21. The device of claim 20, wherein the platform further comprises a bias member configured to positively bias the fiducial marker to the fiducial base.

22. A method of fixing a fiducial marker to an anatomical structure, the fiducial marker being registered in a computer assisted surgical system, comprising the steps of:
a. accessing the anatomical structure through a primary surgical incision;
b. positioning a fixation member on the anatomical structure, the anatomical structure being positioned such that the structure does not disturb surgical approaches within the surgical incision; and
c. sliding a fiducial base into the fixation member, the fiducial base extending the fiducial marker away from the surgical incision such that the fiducial marker is positioned in the field of view of a tracking sensor of the computer assisted surgical system.

23. The method of claim 22, wherein the positioning step further comprises the steps of: a. temporarily affixing the fixation member to the anatomical structure using a first fixation means; b. temporarily sliding the fiducial base into the fixation member to verify the position of the fiducial marker; and c. permanently affixing the fixation member to the anatomical structure using a second fixation means once the position of the fiducial marker has been verified.

24. The method of claim 23, further comprising the step of repositioning the fixation member after the temporarily sliding step when the fiducial marker is not properly positioned because the fixation member is not properly affixed in the temporarily affixing step.

25. (canceled)

26. (canceled)

27. The method of claim 22, further comprising the step of biasing the fiducial base to the fixation member such that the fiducial base is fixed to the fixation member.

28. The method of claim 27 wherein the biasing step further comprises magnetically biasing the fiducial base to the fixation member such that the fiducial base is fixed to the fixation member.

29. A low profile bone fixation member for a fiducial marker, comprising: a. a positioning member configured to secure the bone fixation member to an anatomical structure; b. a guide configured to slidably receive the fiducial marker, the guide being configured to slidably receive the fiducial marker in a direction generally perpendicular to the positioning member; and c. a bias member configured to secure the fiducial marker slidably received by the guide to the guide.

30. The bone fixation member of claim 29, wherein the positioning member comprises a temporary positioning member comprised to temporarily attach the bone fixation member to the anatomical structure.

31. The bone fixation member of claim 30, wherein the temporary positioning member is a spike.

32. (canceled)

33. (canceled)

34. (canceled)

35. (canceled)

36. (canceled)

37. (canceled)

38. (canceled)

39. (canceled)

40. (canceled)
